(19) United States
(12) Patent Application Publication
(10) Pub. No.: US 2020/0393384 A1
(43) Pub. Date: Dec. 17, 2020

(54) pH RESPONSIVE MATERIALS FOR OPTICAL MONITORING OF WOUND STATUS

(71) Applicant: Qualizyme Diagnostics GmbH & Co. KG, Graz (AT)

(72) Inventor: Clemens GAMERITH, Graz (AT)

(21) Appl. No.: 16/901,241

(22) Filed: Jun. 15, 2020

Related U.S. Application Data
(60) Provisional application No. 62/861,747, filed on Jun. 14, 2019.

(51) Int. Cl. G01N 21/80; A61L 15/22; A61L 15/42; C07F 7/08

(52) U.S. Cl. CPC G01N 21/80; C07F 7/0836; A61L 15/42; A61L 15/225

(57) ABSTRACT

The disclosed technology relates to materials for the detection of wounds, e.g., chronic wounds or infected wounds, including compositions, substrates, kits, dressing materials, and articles, and systems containing such compounds. The disclosed technology further relates to methods of using these compositions, kits and systems in the diagnosis and/or detection of chronic or infected wounds based measurement of pH. Additional disclosure relates to methods of characterizing wounds based on pH of wounds or wound fluids and using such information to treat, manage, and follow-up patients suffering from chronic or infected wounds.

### CROSS-REFERENCE TO RELATED APPLICATIONS

The present application claims the benefit of U.S. Provisional Application No. 62/861,747 filed on Jun. 14, 2019, the contents of which are hereby incorporated herein in entirety.

### TECHNICAL FIELD

Embodiments described herein generally relate to wound healing, and in particular to compositions and methods for the detection and treatment of wounds.

### BACKGROUND

In mammals, dermal injury triggers an organized complex cascade of cellular and biochemical events that result in a healed wound. Wound healing is a complex dynamic process that results in the restoration of anatomic continuity and function: an ideally healed wound is one that has returned to normal anatomic structure, function, and appearance. A typical wound heals via a model consisting of four stages—“exudative” phase, proliferative phase, reparative phase and epithelial maturation (Hatz et al., Wound Healing and Wound Management, Springer-Verlag, Munich, 1994) or hemostatic, inflammatory, proliferative and remodeling phase (Nwomeh et al., Clin. Plast. Surg. 1998, 25, 341). The inflammatory phase is particularly important to the wound healing process, wherein biochemical reactions at the wound situs facilitate healing but also cause tissue breakdown due to production of excess proteases.

Pathogenic infection is one of the most common impediments to wound healing. A progressive worsening of a clean wound to a colonized wound is often associated with increased bioburden imposed by pathogenic microorganisms. See, Ovington et al., Ostomy Wound Management, 49.7A:8-12, 2003. An infected wound is an intermediate stage that is characterized by clinical signs of infection such as yellow appearance, soreness, redness, oozing pus, while a colonized wound is characterized by chronic pathogenic infection and is difficult to heal. Infection of the wound may also arrest the healing process. For example, pathogens in a wound can produce toxins (e.g., Clostridium species), generate noxious metabolites like ammonia that raise pH (e.g., Proteus species), activate or produce tissue lytic enzymes like proteases, or promote tissue invasion, thereby leading to an increase in the size or seriousness of the wound.

In order to keep the chronicity of wounds in check, a variety of assessment techniques and/or tools are employed in the clinical and veterinary setting. Current methods of assessing an infected wound are based primarily on assaying for a variety of parameters associated with the wound. For instance, a wound may be assessed visually, length and depth measurements may be taken, digital photography may be used where available to track the visual condition and size of a wound (Krasner et al., supra). In clinical practice, diagnosis of infection is based on measurement of secondary parameters, such as, odor, presence of local pain, heat, swelling, discharge, and redness. Many of these clinical indicators, such as inflammation and discharge have a low predictive value of infection in wounds. In other instances, the number(s) and type(s) of pathogenic flora at the wound situs may be determined using laboratory and/or clinical diagnostic procedures. Swabbing of a wound followed by microbiology testing in the hospital laboratory is an option for confirmation of bacterial colonization and identification of the strains associated with infection, thus allowing for the prescription of correct antibiotic course. However, this process is time consuming and labor intensive. Delay in diagnosis of infection can delay the administration of antibiotics and may increase the risk of developing sepsis.

One of the biggest drawbacks associated with existing clinical diagnostics is a lag associated with the onset of infection and the timing of detection. For instance, positive identification of infection using swabbing procedures often depends on attainment of a “critical mass” of microorganisms at the wound site and so early detection cannot be made until a detectable level is reached. Also, the swabs may be contaminated with the flora of the surrounding tissue, thereby complicating the diagnostic procedure. Other drawbacks include, e.g., sampling errors, delays in transport of the swabs, errors in analytical procedures, and/or errors in reporting. See, the review by Bowler et al., Clin Microbiol Rev. 14(2): 244-269, 2001.

There is therefore an imminent but unmet need for diagnostic reagents and methods that enable early diagnosis of clinical infection, preferably, which permit clinical diagnosis prior to manifestation of clinical symptoms of infection. There is also a need for compositions and methods that would assist in predicting clinical infection of a wound prior to the manifestation of clinical symptoms. Such a prognostic aid would allow early intervention with suitable treatment (e.g., antimicrobial treatment) before the wound is exacerbated and surgery or other drastic intervention is required to prevent further infection. Additionally, if clinicians could respond to wound infection as early as possible, the infection could also be treated with minimal antibiotic usage. This would reduce the need for hospitalization and would reduce the risk of secondary infections, e.g., as a result of contact with other diseased subjects.

### SUMMARY

The technology disclosed herein provides for compositions and methods of detecting infected and/or chronic wounds. The disclosed technology improves upon existing assays by increasing the speed of detection of infected wounds in situ and in real-time, as well as providing a simple visual readout of elevated pH of a wound or wound fluid. The assays and methods described herein are partly based on the use of specific reagents that detect biomarkers which are present in infected or chronic wounds. Furthermore, the novel probes and the assay techniques based thereon are capable of detecting and characterizing various types of wounds. Finally, the reagents of the disclosed technology may be used together with therapeutic molecules such as antibiotics, antifungal agents, etc. to monitor and evaluate treatment and management of chronic wounds.

Embodiments described herein are based, in part on the discovery that elevated pH values of about 7.0 and above are associated with non-healing wounds or infected burns. This suggests that elevated pH values in wounds or wound fluids can be used as one of a set of potential markers for wound infection, enabling early diagnosis of wound infection and timely appropriate treatment. Certain embodiments described herein utilize a visual color change that can be viewed in the presence of wound fluid, allowing for facile and rapid detection of elevated wound pH.

Accordingly, various embodiments described herein utilize the detection of elevated pH in a biological sample of interest, for example, wound tissue. Increased pH in the wound fluid, therefore, corresponds to a heightened bacterial challenge and a manifestation of disturbed host/bacteria equilibrium in favor of the invasive bacteria.

Disclosed herein are pH responsive materials, comprising; an anchor material; a linking moiety; and a pH responsive element. In some instances, the linking moiety connects the anchor material and the pH responsive element via covalent bonds. In other instances, the anchor material comprises OH-bearing surfaces. In yet other embodiments, the anchor material comprises a polysaccharide, a cellulose, or a monomer thereof, an oligomer thereof, a derivative thereof, a mixture or a combination thereof. In some instances, the anchor material comprises a polysaccharide selected from hydroxypropyl methylcellulose (HPMC), hydroxyethyl cellulose, hydroxymethyl cellulose, D-galactopyranoside, or a derivative thereof. In still other instances, the anchor material is a solid support. In some instances, the solid support is cotton, paper, filter paper, a chip, a pin, a slide, a membrane, a bead, a cotton swab, a wound dressing, or a particle. In yet other instances, the solid support is a cellulose particle. In yet other instances, the cellulose particle has a particle size of up to about 1, 2, 3, 4, 5, 6, 7, 8, 9, 10, 15, 20, 25, 30, 35, 40, 45, 50, 60, 70, 80, 90, or 100 μm. In certain instances, the cellulose particle has a particle size of about 20 μm. In some instances, the solid support is a cotton swab. In other instances, the cotton swab is suitable for use in a wound.

In some instances, the linking moiety of the pH responsive materials disclosed herein is a siloxane monomer or polymer thereof. In some instances, the siloxane monomer or polymer thereof comprises an epoxide functional group. In other instances, the siloxane monomer or polymer thereof comprises one or more monomers selected from GPTMS, Diethoxy(3-glycidyloxypropyl)methylsilane, 3-Glycidoxypropyldimethoxymethylsilane, epoxycyclohexyl)ethyltriethoxysilane, 2-(3,4-epoxycyclohexyl)ethyltrimethoxysilane, or combinations thereof. In yet other instances, the siloxane monomer is GPTMS. In some instances, the pH responsive element presents a visible color change at a pH of about 7.0. In some instances, the visible color change is from yellow to blue. In some instances, the pH responsive element is selected from thymol blue, bromophenol blue, bromocresol green, bromocresol purple, bromothymol blue, phenol red, napththolpthalein, cresol red, cresolpthalein, phenolphthalein, or thymolpthalein. In other embodiments, the pH responsive element is a pH indicator comprising multiple phenol functional groups. In yet other embodiments, the pH responsive element is bromocresol purple. In some instances, in the pH responsive element is functionalized onto the linking moiety through a reaction of a phenol functional group of the pH responsive element with an epoxide functional group of the linking moiety.

Also discussed herein are pH responsive materials having the structure:

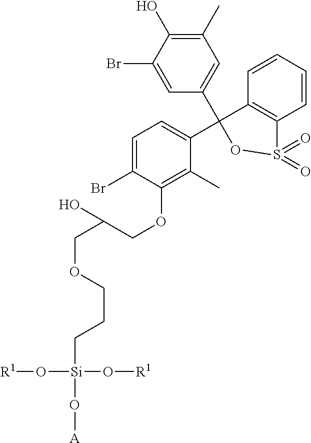

Wherein A is cellulose or a cellulose polymer; and each R¹ is independently H or one or more functionalized or non-functionalized GPTMS monomers or a combination thereof.

In some embodiments, the cellulose particle(s) of the pH responsive materials disclosed herein is incorporated into a medical or diagnostic device. In some embodiments, the cellulose particle is incorporated into a medical or diagnostic device by spraying, printing, or depositing the cellulose particle. In yet other embodiments, the cellulose particle is sprayed, printed, or deposited on a solid phase of the medical or diagnostic device. In some embodiments, the solid phase of the medical or diagnostic device comprises a dressing, a wound dressing, a bandage, filter paper, or a test strip.

Also disclosed herein are methods of making pH responsive materials disclosed herein comprising the steps of: dissolving a linking moiety in an acidic solution; immersing an anchor material in the acidic solution; drying the anchor material; and soaking the anchor material in a solution comprising a pH responsive element. In some embodiments, the linking moiety is a silane reagent. In yet other embodiments, the silane reagent comprises an epoxide functional group. In yet other instances, the silane reagent is selected from GPTMS, Diethoxy(3-glycidyloxypropyl)methylsilane, 3-Glycidoxypropyldimethoxymethylsilane, epoxycyclohexyl)ethyltriethoxysilane, 2-(3,4-epoxycyclohexyl)ethyltrimethoxysilane, or combinations thereof. In some embodiments, the silane reagent is GPTMS. In still other instances, the acidic solution comprises an organic acid. In some embodiments, the acidic solution comprises acetic acid. In yet other instances, the acetic acid is at a concentration of about 57 μM. In some embodiments, the anchor material comprises OH-bearing surfaces. In some embodiments, the anchor material comprises a polysaccharide, a cellulose, or a monomer thereof, an oligomer thereof, a derivative thereof, a mixture or a combination thereof. In yet other embodiments, the anchor material comprises a polysaccharide selected from hydroxypropyl methylcellulose (HPMC), hydroxyethyl cellulose, hydroxymethyl cellulose, D-galactopyranoside, or a derivative thereof. In some embodiments, the anchor material is a solid support. In other instances, the solid support is selected from cotton, paper, filter paper, a cotton swab, a wound dressing, or a cellulose particle. In some embodiments, the solid support is a cellulose particle. In some instances, the cellulose particle has a particle size of up to about 1, 2, 3, 4, 5, 6, 7, 8, 9, 10, 15, 20, 25, 30, 35, 40, 45, 50, 60, 70, 80, 90, or 100 μm. In other instances, the cellulose particle has a particle size of about 20 μm. In some instances, the solid support is a cotton swab. In other instances, the cotton swab is suitable for using in a wound. In other embodiments, the anchor material is immersed in the acidic solution for at least about 5, 10, 15, 20, 25, or 30 minutes. In some instances, the acidic solution is at a temperature of at least about 60, 70, 80, 90, 100, 110, or 120° C. In other embodiments, the pH responsive element is a pH indicator. In some instances, the pH indicator comprises multiple phenol functional groups. In some instances, the pH indicator is selected from thymol blue, bromophenol blue, bromocresol green, bromocresol purple, bromothymol blue, phenol red, napththolpthalein, cresol red, cresolpthalein, phenolphthalein, or thymolpthalein. In other embodiments, the pH indicator is bromocresol purple. In some instances, the pH indicator is functionalized onto the linking moiety through a reaction of a phenol functional group of the pH indicator with an epoxide functional group of the linking moiety.

Also disclosed herein are methods of diagnosing a status of a wound, comprising the steps of: contacting the wound or a wound fluid with a pH responsive material as disclosed herein, and detecting a color change of the pH responsive material.

Also disclosed herein are methods of treating an infected wound comprising the steps of: contacting the wound or a wound fluid with a pH responsive material as disclosed herein; detecting a color change of the pH responsive material; and administering a treatment for an infection.

### DESCRIPTION OF THE FIGURES

FIG. 1 illustrates a schematic representation of the immobilization reaction of the pH indicator, bromocresol purple (BCP), with GPTMS onto materials containing hydroxyl groups on the surfaces.

FIG. 2A illustrates the chemical formula of BCP.

FIG. 2B illustrates the chemical formula of GPTMS.

FIG. 2C illustrates the proposed chemical structure of the reaction product on paper.

FIG. 3A illustrates the absorbance spectra of functionalized BCP (FBCP) under acidic (pH 4.0) and alkaline (pH 10.0) conditions. The background absorbance of the cellulose itself was subtracted for a better comparability of the graphs in FIG. 3A and FIG. 3B.

FIG. 3B illustrates the absorbance spectra of BCP under acidic (pH 4.0) and alkaline (pH 10.0) conditions.

FIG. 4 illustrates color change of immobilized bromocresol purple (FBCP) and free BCP over a pH range of 5.0 to 8.5, as well as a schematic comparison to infection risk status.

FIG. 5 illustrates pH-responsive color changes of FBCP shown as b* values including standard deviation (pH range from 5.0-8.0).

FIG. 6 illustrates normalized FTIR spectra of a cellulose filter and a cellulose filter containing FBCP.

FIG. 7 illustrates receiver operator characteristics curve for the pH responsive FBCP in an ex-vivo setup (156 patients) for the detection of infection (Sensitivity 53.8%; Specificity 78.5%; grey line reference line; black line pH).

FIG. 8A depicts settled particles (pH 5.0, 6.0, 7.0, 8.0) different pH values of the pH sensitive material.

FIG. 8B depicts particles in suspension (pH 5.0, 6.0, 7.0, 8.0) of the pH sensitive material.

FIG. 8C depicts modified cotton swabs (initial color, pH 5.0, 6.0, 7.0, 8.0) of the pH sensitive material.

FIG. 8D depicts the integration of FBCP into a dressing (pH 5.0, 8.0).

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

Overview

Provided herein are compositions and systems for the therapy and diagnosis of wounds and wound management, wherein the compositions, when in use, indicate the presence of elevated pH in a wound in situ.

As used herein, a “wound” refers to physical disruption of the continuity or integrity of tissue structure. “Wound healing” refers to the restoration of tissue integrity. It will be understood that this can refer to a partial or a full restoration of tissue integrity. Treatment of a wound thus refers to the promotion, improvement, progression, acceleration, or otherwise advancement of one or more stages or processes associated with the wound healing process.

The wound may be acute or chronic. Chronic wounds, including pressure sores, venous leg ulcers and diabetic foot ulcers, can simply be described as wounds that fail to heal. Whilst the exact molecular pathogenesis of chronic wounds is not fully understood, it is acknowledged to be multi-factorial. As the normal responses of resident and migratory cells during acute injury become impaired, these wounds are characterized by a prolonged inflammatory response, defective wound extracellular matrix (ECM) remodeling and a failure of re-epithelialization.

The wound may be any internal wound, e.g., where the external structural integrity of the skin is maintained, such as in bruising or internal ulceration, or external wounds, particularly cutaneous wounds, and consequently the tissue may be any internal or external bodily tissue. In one embodiment, the tissue is skin (such as human skin), i.e. the wound is a cutaneous wound, such as a dermal or epidermal wound.

The human skin is composed of two distinct layers, the epidermis and the dermis, below which lies the subcutaneous tissue. The primary functions of the skin are to provide protection to the internal organs and tissues from external trauma and pathogenic infection, sensation and thermoregulation. The skin tissue of most mammals is structured similarly.

The outermost layer of skin, the epidermis, is approximately 0.04 mm thick, is avascular, is comprised of four cell types (keratinocytes, melanocytes, Langerhans cells, and Merkel cells), and is stratified into several epithelial cell layers. The inner-most epithelial layer of the epidermis is the basement membrane, which is in direct contact with, and anchors the epidermis to, the dermis. All epithelial cell division occurring in skin takes place at the basement membrane. After cell division, the epithelial cells migrate towards the outer surface of the epidermis. During this migration, the cells undergo a process known as keratinization, whereby nuclei are lost and the cells are transformed into tough, flat, resistant non-living cells. Migration is completed when the cells reach the outermost epidermal structure, the stratum corneum, a dry, waterproof squamous cell layer which helps to prevent desiccation of the underlying tissue. This layer of dead epithelial cells is continuously being sloughed off and replaced by keratinized cells moving to the surface from the basement membrane. Because the epidermal epithelium is avascular, the basement membrane is dependent upon the dermis for its nutrient supply.

The dermis is a highly vascularized tissue layer supplying nutrients to the epidermis. In addition, the dermis contains nerve endings, lymphatics, collagen protein, and connective tissue. The dermis is approximately 0.5 mm thick and is composed predominantly of fibroblasts and macrophages. These cell types are largely responsible for the production and maintenance of collagen, the protein found in all animal connective tissue, including the skin. Collagen is primarily responsible for the skin's resilient, elastic nature. The subcutaneous tissue, found beneath the collagen-rich dermis, provides for skin mobility, insulation, calorie storage, and blood to the tissues above it.

Wounds can be classified in one of two general categories, partial thickness wounds or full thickness wounds. A partial thickness wound is limited to the epidermis and superficial dermis with no damage to the dermal blood vessels. A full thickness wound involves disruption of the dermis and extends to deeper tissue layers, involving disruption of the dermal blood vessels. The healing of the partial thickness wound occurs by simple regeneration of epithelial tissue. Wound healing in full thickness wounds is more complex. Cutaneous wounds contemplated herein may be either partial thickness or full thickness wounds.

Wounds contemplated herein include cuts and lacerations, surgical incisions or wounds, punctures, grazes, scratches, compression wounds, abrasions, friction wounds (e.g., nappy rash, friction blisters), decubitus ulcers (e.g., pressure or bed sores); thermal effect wounds (burns from cold and heat sources, either directly or through conduction, convection, or radiation, and electrical sources), chemical wounds (e.g. acid or alkali burns) or pathogenic infections (e.g., viral, bacterial or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, immune response conditions, e.g., psoriasis and eczema, stomach or intestinal ulcers, oral wounds, including a ulcers of the mouth, damaged cartilage or bone, amputation wounds and corneal lesions.

In some instances, the pH of a wound can influence many factors of wound healing, such as angiogenesis, protease activity, oxygen release, and bacterial toxicity. Chronic non-healing wounds may have an elevated alkaline environment. As the wound progresses towards healing, the pH of the wound moves to neutral and then becomes acidic. Monitoring of the pH of the wound may provide a method to assess the condition of the wound (e.g., infection or no infection) and aid in determining a wound's response to treatment.

### Definitions

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

“Substantially” or “essentially” means nearly totally or completely, for instance, 80%-95% or greater of some given quantity, e.g., at least 85%, at least 87%, at least 88%, at least 89%, at least 90%, at least 91%, at least 92%, at least 93%, at least 94%, at least 95%, at least 96%, at least 97%, at least 98%, at least 99%, at least 99.9%, or more % by weight or volume or any other parameter being measured. “Substantially free” means nearly totally or completely absent of some given quantity such as being present at a level of less than about 1% to about 20% of some given quantity, e.g., less than 10%, less than 9%, less than 8%, less than 7%, less than 6%, less than 5%, less than 4%, less than 3%, less than 2%, less than 1%, less than 0.5%, less than 0.1%, or less % by weight or volume or any other parameter being measured. In some embodiments, “substantially free” means presence at a level of less than or equal to 1-5% by weight of the pharmaceutical composition.

pH Responsive Materials and Compositions Thereof

Embodiments herein provide pH responsive materials, which may be used to diagnose and/or treat chronic wounds. In some embodiments, the pH responsive materials and compositions thereof are used in methods to detect the pH of a mammalian wound. In some embodiments, the pH responsive materials and compositions thereof are used in methods to diagnose an infected wound in a mammal. In some embodiments, the pH responsive materials and compositions thereof described herein are used in methods to treat a wound in a mammal. In further embodiments, the pH responsive materials and compositions thereof described herein are used in methods to treat an infected or a chronic wound in a mammal.

In some embodiments, provided herein is a pH responsive material capable of detecting pH of a body fluid, the pH responsive material comprising: an anchor material, linking moiety, and pH responsive element. In certain embodiments, the linking moiety connects the anchor material and the pH responsive element via one or more bonds. In certain embodiments, the linking moiety connects the anchor material and the pH responsive element via covalent bonds. In certain embodiments, the linking moiety connects the anchor material and the pH responsive element via ionic bonds. In certain embodiments, the linking moiety connects the anchor material and the pH responsive element via hydrophobic interactions.

In certain embodiments, the linking moiety may be naturally present in the anchor material. In certain embodiments, the linking moiety is introduced in the anchor material by chemical modification. In certain embodiments, the linking moiety may be naturally present in the pH responsive element. In certain embodiments, the linking moiety is introduced in the pH responsive element by chemical modification. In certain embodiments, the linking moiety is a distinct molecule from the anchor material and the pH responsive element.

Anchor Materials

In some embodiments of the pH responsive material, the anchor material comprises OH-bearing surfaces. OH-bearing surfaces are materials that have one or more free hydroxyl groups that are accessible to interact with chemical reagents. Non-limiting examples of anchor materials comprising OH-bearing surfaces include polysaccharides, cellulose, monomers thereof, oligomers thereof, derivatives thereof, and mixtures or combinations thereof. In certain embodiments, OH-bearing surfaces are utilized to bond a linking moiety with the anchor material.

In certain embodiments, the anchor material comprises a polysaccharide, a cellulose, or a monomer thereof, an oligomer thereof, a derivative thereof, or a mixture or a combination thereof. In certain embodiments, the anchor material is cellulose. In certain embodiments, the anchor material is a cellulose polymer.

In certain embodiments, the anchor material is a polysaccharide. In certain embodiments, the anchor material is a polysaccharide derivative. Non-limiting examples of polysaccharide derivatives include hydroxyethyl cellulose (HEC), hydroxypropyl cellulose (HPC), ethyl hydroxyethyl cellulose (EHEC), carboxymethyl cellulose, carboxymethyl hydroxyethyl cellulose (CMHEC), hydroxypropyl hydroxyethyl cellulose (HPHEC), methyl cellulose (MC), methyl hydroxypropyl cellulose (MHPC), methyl hydroxyethyl cellulose (MHEC), carboxymethyl cellulose (CMC), hydroxypropyl methylcellulose acetate succinate (HPMC-AS), hydrophobically modified hydroxyethyl cellulose (hmHEC), hydrophobically modified hydroxypropyl cellulose (hmHPC), hydrophobically modified ethyl hydroxyethyl cellulose (hmEHEC), hydrophobically modified carboxymethyl hydroxyethyl cellulose (hmCMHEC), hydrophobically modified hydroxypropyl hydroxyethyl cellulose (hmHPHEC), hydrophobically modified methyl cellulose (hmMC), hydrophobically modified methyl hydroxypropyl cellulose (hmMHPC), hydrophobically modified methyl hydroxyethyl cellulose (hmMHEC), hydrophobically modified carboxymethyl methyl cellulose (hmCMMC), sulfoethyl cellulose (SEC), hydroxyethyl sulfoethyl cellulose (HESEC), hydroxypropyl sulfoethyl cellulose (HPSEC), methyl hydroxyethyl sulfoethylcellulose (MHESEC), methyl hydroxypropyl sulfoethyl cellulose (MHPSEC), hydroxyethyl hydroxypropyl sulfoethyl cellulose (HEHPSEC), carboxymethyl sulfoethyl cellulose (CMSEC), hydrophobically modified sulfoethyl cellulose (hmSEC), hydrophobically modified hydroxyethyl sulfoethyl cellulose (hmHESEC), hydrophobically modified hydroxypropyl sulfoethyl cellulose (hmHPSEC) or hydrophobically modified hydroxyethyl hydroxypropyl sulfoethyl cellulose (hmHEHPSEC). In certain embodiments, the anchor material comprises a polysaccharide selected from hydroxypropyl methylcellulose (HPMC), hydroxyethyl cellulose, hydroxymethyl cellulose, D-galactopyranoside, or a derivative thereof. In certain embodiments, the anchor material comprises from hydroxypropyl methylcellulose (HPMC). In certain embodiments, the anchor material comprises from hydroxyethyl cellulose. In certain embodiments, the anchor material comprises from hydroxymethyl cellulose. In certain embodiments, the anchor material comprises D-galactopyranoside.

In certain embodiments, the anchor material is a solid support. Useful solid supports include natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

A solid support may take the form of a desired structure. Non-limiting examples of solid supports include cotton, paper, filter paper, chips, pins, slides, membranes, beads, swabs, wound dressings, and particles. In certain embodiments, as described herein, is a pH responsive material wherein the anchor material is a solid support, wherein the solid support t is cotton, paper, filter paper, a chip, a pin, a slide, a membrane, a bead, a swab, a wound dressing, or a particle. In certain embodiments, the solid support is cotton. In certain embodiments, the solid support is a swab. In certain embodiments, the solid support is paper. In certain embodiments, the solid support is filter paper. In certain embodiments, the solid support is a chip. In certain embodiments, the solid support is a pin. In certain embodiments, the solid support is a slide. In certain embodiments, the solid support is a membrane. In certain embodiments, the solid support is a bead. In certain embodiments, the solid support is a wound dressing. In certain embodiments, the solid support is a particle.

In certain embodiments, as described herein, is a pH responsive material comprising a solid support anchor material, wherein the solid support anchor material is a cellulose particle. In certain embodiments, the cellulose particle has a particle size of up to about 1, 2, 3, 4, 5, 6, 7, 8, 9, 10, 15, 20, 25, 30, 35, 40, 45, 50, 60, 70, 80, 90, or 100 μm. In certain embodiments, the cellulose particle has a particle size of up to about 1 μm. In certain embodiments, the cellulose particle has a particle size of up to about 2 μm. In certain embodiments, the cellulose particle has a particle size of up to about 3 μm. In certain embodiments, the cellulose particle has a particle size of up to about 4 μm. In certain embodiments, the cellulose particle has a particle size of up to about 5 μm. In certain embodiments, the cellulose particle has a particle size of up to about 6 μm. In certain embodiments, the cellulose particle has a particle size of up to about 7 μm. In certain embodiments, the cellulose particle has a particle size of up to about 8 μm. In certain embodiments, the cellulose particle has a particle size of up to about 9 μm. In certain embodiments, the cellulose particle has a particle size of up to about 10 μm. In certain embodiments, the cellulose particle has a particle size of up to about 15 μm. In certain embodiments, the cellulose particle has a particle size of up to about 20 μm. In certain embodiments, the cellulose particle has a particle size of up to about 25 μm. In certain embodiments, the cellulose particle has a particle size of up to about 30 μm. In certain embodiments, the cellulose particle has a particle size of up to about 35 μm. In certain embodiments, the cellulose particle has a particle size of up to about 40 μm. In certain embodiments, the cellulose particle has a particle size of up to about 45 μm. In certain embodiments, the cellulose particle has a particle size of up to about 50 μm. In certain embodiments, the cellulose particle has a particle size of up to about 60 μm. In certain embodiments, the cellulose particle has a particle size of up to about 70 μm. In certain embodiments, the cellulose particle has a particle size of up to about 80 μm. In certain embodiments, the cellulose particle has a particle size of up to about 90 μm. In certain embodiments, the cellulose particle has a particle size of up to about 100 μm.

In certain embodiments is a pH responsive material comprising a solid support anchor material, wherein the solid support anchor material is a cotton swab. In certain embodiments, the cotton swab is suitable for use in a wound. In certain embodiments, the cotton swab is sterile.

In certain embodiments is a pH responsive material comprising a solid support anchor material, wherein the solid support anchor material is filter paper. In certain embodiments, the filter paper is incorporated into a wound dressing. In certain embodiments, the filter paper is incorporated into a wound dressing between an adhesive layer and a non-adhesive layer. In certain embodiments, the filter paper incorporated into a wound dressing such that it will become wet with wound fluid after application to a wound.

In some embodiments, the pH responsive material is printed on or in a support material. In some embodiments, the pH responsive material bonds to a support material through the anchor material of the pH responsive material. In some embodiments, the support material is filter paper or a woven or non-woven material that is capable of being wet by a wound fluid and which displays capillary action. In some embodiments, the pH responsive material is chemically bonded onto or into a support material such as filter paper or a woven or non-woven material that is capable of being wet by a wound fluid and which displays capillary action that is similar in all dimensions. In some embodiments, pH responsive material is ionically bound onto or into a support material such as filter paper or a woven or non-woven material that is capable of being wet by a wound fluid and which displays capillary action. In some embodiments, the pH responsive material is covalently bound onto or into a support material such as filter paper or a woven or non-woven material that is capable of being wet by a wound fluid and which displays capillary action. Support material includes, but is not limited to, cellulose, polyamide, polyester, polyacrylate and other similar polymers that are useful as fibers. In some embodiments, the support material is cellulose. In some embodiments, the support material is polyamide. In some embodiments, the support material is polyester. In some embodiments, the support material is polyacrylate.

Linking Moieties

In some embodiments, the pH responsive material comprises a linking moiety. The linking moiety may be attached to the anchor material covalently or non-covalently. As is understood in the art, covalent bonds involve sharing of electrons. In contrast, non-covalent bonds may include, for example, ionic interactions, electrostatic interactions, hydrogen bonding interactions, physiochemical interactions, van der Waal forces, Lewis-acid/Lewis-base interactions, or combinations thereof. Particularly, the linker is attached or conjugated to the anchor material via covalent interaction.

In certain embodiments, the linking moiety is a siloxane monomer or polymer thereof. As is understood in the art, a siloxane is a functional group with an Si—O—Si linkage. A siloxane polymer is a molecule with the form HO—(SiR₂—O)ₙ—H, wherein n is any number and each R is independently bonds to any other atom. As used herein, the term “siloxane monomer” refers to a siloxane polymer with the form HO—(SiR₂—O)ₙ—H, wherein n is 1. In certain embodiments, the siloxane monomer or polymer linking moiety is formed by a reaction of a silane or oxysilane reagent. As is understood in the art, a silane is a compound of the SiR₄, wherein each R group is independently another atom. An oxysilane is a silane which has one or more Si—O—R bonds, wherein R is not silicon.

In certain embodiments, the linking moiety is formed from a reaction of an OH-bearing surface and a silane reagent. In certain embodiments, the linking moiety formed from a reaction of an OH-bearing surface and a silane reagent is a siloxane monomer. In certain embodiments, the silane reagent further reacts with additional silane reagents to form a siloxane polymer. In certain embodiments, the silane reagent does not react with additional silane reagents. In certain embodiments wherein the silane reagent does not react with additional silane reagents, the linking moiety is a silane monomer. In certain embodiments, the silane reagent is an oxysilane. In certain embodiments, the linking moiety comprises a covalent bond between the silane reagent and an OH group on the OH-bearing surface.

In certain embodiments, the linking moiety is a siloxane monomer or polymer thereof. In certain embodiments, the siloxane monomer or polymer thereof comprises a reactive functional group. In certain embodiments, the reactive functional group is useful for functionalization with a pH responsive element. Non-limiting examples of such reactive functional groups include epoxides, alkyl halides, activated esters (e.g. N-hydroxysuccinimde), and maleimides.

In certain embodiments, the siloxane monomer or polymer comprises an epoxide functional group. In certain embodiments, the siloxane monomer or polymer thereof comprises one or more monomers selected from (3-glycidylpropyl)trimethoxysilane (GPTMS), Diethoxy(3-glycidyloxypropyl)methylsilane, 3-Glycidoxypropyldimethoxymethylsilane, epoxycyclohexyl)ethyltriethoxysilane, 2-(3,4-epoxycyclohexyl)ethyltrimethoxysilane, or combinations thereof. In certain embodiments, the siloxane monomer is GPTMS. In certain embodiments, the siloxane monomer is Diethoxy(3-glycidyloxypropyl)methylsilane. In certain embodiments, the siloxane monomer is 3-Glycidoxypropyldimethoxymethylsilane. In certain embodiments, the siloxane monomer is 2-(3,4-epoxycyclohexyl)ethyltriethoxysilane. In certain embodiments, the siloxane monomer is 2-(3,4-epoxycyclohexyl)ethyltrimethoxysilane.

In certain embodiments, the linking moiety is functionalized with a pH responsive element. In certain embodiments, the linking moiety is functionalized with a pH responsive element by one or more covalent bonds. In certain embodiments, the one or more covalent bonds are formed through a reaction of the pH responsive element and the reactive functional group of the linking moiety. In certain embodiments, the one or more covalent bonds are formed through a reaction of the pH responsive element and an epoxide functional group of the linking moiety. In certain embodiments, the one or more covalent bonds are formed through a reaction of a phenol functional group of the pH responsive element with an epoxide functional group of the linking moiety.

In certain embodiments, at least 10%, 20%, 30%, 40%, 50%, 60%, 70%, 80%, 90%, or 100% of the linking moieties are functionalized with a pH responsive element. In certain embodiments, at least 10% of the linking moieties are functionalized with a pH responsive element. In certain embodiments, at least 20% of the linking moieties are functionalized with a pH responsive element. In certain embodiments, at least 30% of the linking moieties are functionalized with a pH responsive element. In certain embodiments, at least 40% of the linking moieties are functionalized with a pH responsive element. In certain embodiments, at least 50% of the linking moieties are functionalized with a pH responsive element. In certain embodiments, at least 60% of the linking moieties are functionalized with a pH responsive element. In certain embodiments, at least 70% of the linking moieties are functionalized with a pH responsive element. In certain embodiments, at least 80% of the linking moieties are functionalized with a pH responsive element. In certain embodiments, at least 90% of the linking moieties are functionalized with a pH responsive element. In certain embodiments, 100% of the linking moieties are functionalized with a pH responsive element.

pH Responsive Elements

In some embodiments, the pH responsive materials comprise a pH responsive element. In certain embodiments, the pH responsive element presents a visible color change. In one embodiment, the pH responsive element presents a visible color change at alkaline pH, e.g., a pH=7.2-9.5; pH=7.2-9.0; pH=7.2-8.5; pH=7.2-8.0; pH=7.5-8.5; pH=7.5-9.0; pH=8.0-9.0. In other embodiments, the pH responsive element presents a visible color change at pH=7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5, or 0.1 increments thereof.

In some embodiments, the pH responsive element presents a visible color change at neutral pH range, e.g., at pH=6.9, 7.0, or 7.1, or 0.05 increments thereof. In certain embodiments, the pH responsive element presents a color change at a pH of about 7.0.

In some embodiments, the pH responsive element presents a visible color change at acidic pH, e.g., pH=4.5-6.8; pH=4.5-6.5; pH=5.0-6.8; pH=5.4-6.8; pH=5.4-6.5. In other embodiments, the pH responsive element presents a visible color change at pH=4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or 0.1 increments thereof.

In certain embodiments, the visible color change is discernable from the color of the wound or wound fluid. In certain embodiments, the visible color change is from yellow to blue.

In certain embodiments, the pH responsive element is a pH indicator. In certain embodiments, the pH indicator comprises multiple phenol functional groups. In certain embodiments, the pH indicator is selected from thymol blue, bromophenol blue, bromocresol green, bromocresol purple, bromothymol blue, phenol red, napththolpthalein, cresol red, cresolpthalein, phenolphthalein, or thymolpthalein. In certain embodiments, the pH indicator is thymol blue. In certain embodiments, the pH indicator is bromophenol blue. In certain embodiments, the pH indicator is bromocresol green. In certain embodiments, the pH indicator is bromocresol purple. In certain embodiments, the pH indicator is thymol blue. In certain embodiments, the pH indicator is bromothymol blue. In certain embodiments, the pH indicator is phenol red. In certain embodiments, the pH indicator is napththolpthalein. In certain embodiments, the pH indicator is cresol red. In certain embodiments, the pH indicator is cresolpthalein. In certain embodiments, the pH indicator is phenolphthalein. In certain embodiments, the pH indicator is thymolpthalein.

pH Responsive Material Structure

In certain embodiments, the pH responsive material has the structure

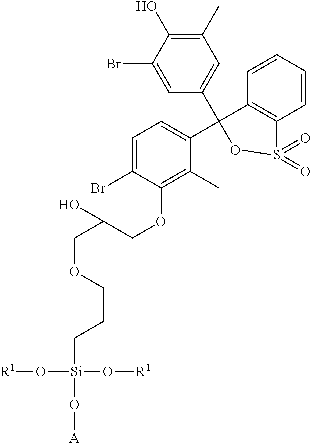

In certain embodiments, A is an anchor material as described herein. In certain embodiments, A is cellulose or a cellulose polymer. In certain embodiments, each R¹ is independently H or one or more functionalized or non-functionalized GPTMS monomers or a combination thereof. In certain embodiments, each R¹ is H. In certain embodiments, each R¹ is a functionalized GPTMS monomer. In certain embodiments, each R¹ is a functionalized GPTMS polymer. In certain embodiments, each R¹ is a non-functionalized GPTMS monomer. In certain embodiments, each R¹ is a non-functionalized GPTMS polymer. In certain embodiments, each R¹ is a mixture of functionalized and non-functionalized GPTMS monomers. In certain embodiments, one R¹ is H and the other R¹ is functionalized GPTMS monomer, a non-functionalized GPTMS monomer, a functionalized GPTMS polymer, a non-functionalized GPTMS polymer, or a mixture functionalized and non-functionalized GPTMS monomers.

In certain embodiments, as described herein, A is a cellulose particle. In certain embodiments, the cellulose particle has a particle size of up to about 1, 2, 3, 4, 5, 6, 7, 8, 9, 10, 15, 20, 25, 30, 35, 40, 45, 50, 60, 70, 80, 90, or 100 μm. In certain embodiments, the cellulose particle has a particle size of up to about 1 μm. In certain embodiments, the cellulose particle has a particle size of up to about 2 μm. In certain embodiments, the cellulose particle has a particle size of up to about 3 μm. In certain embodiments, the cellulose particle has a particle size of up to about 4 μm. In certain embodiments, the cellulose particle has a particle size of up to about 5 μm. In certain embodiments, the cellulose particle has a particle size of up to about 6 μm. In certain embodiments, the cellulose particle has a particle size of up to about 7 μm. In certain embodiments, the cellulose particle has a particle size of up to about 8 μm. In certain embodiments, the cellulose particle has a particle size of up to about 9 μm. In certain embodiments, the cellulose particle has a particle size of up to about 10 μm. In certain embodiments, the cellulose particle has a particle size of up to about 15 μm. In certain embodiments, the cellulose particle has a particle size of up to about 20 μm. In certain embodiments, the cellulose particle has a particle size of up to about 25 μm. In certain embodiments, the cellulose particle has a particle size of up to about 30 μm. In certain embodiments, the cellulose particle has a particle size of up to about 35 μm. In certain embodiments, the cellulose particle has a particle size of up to about 40 μm. In certain embodiments, the cellulose particle has a particle size of up to about 45 μm. In certain embodiments, the cellulose particle has a particle size of up to about 50 μm. In certain embodiments, the cellulose particle has a particle size of up to about 60 μm. In certain embodiments, the cellulose particle has a particle size of up to about 70 μm. In certain embodiments, the cellulose particle has a particle size of up to about 80 μm. In certain embodiments, the cellulose particle has a particle size of up to about 90 μm. In certain embodiments, the cellulose particle has a particle size of up to about 100 μm.

In certain embodiments, as described herein, A is a cotton swab. In certain embodiments, the cotton swab is suitable for use in a wound. In certain embodiments, the cotton swab is sterile.

Compositions

Embodiments described herein further relate to compositions containing the pH responsive materials described herein. Once formulated, the composition of the pH responsive materials may be further into desired form, e.g., gels, balms, lotions, cream, paste, ointments, etc. using conventional methods, e.g., using carriers, gelling agents, emollients, surfactants, humectants, viscosity enhancers, emulsifiers, etc. See, e.g., WO 2013/004953.

Carriers for use in the composition may include, but are not limited to, water, glycerin, diglycerin, glycerin derivatives, glycols, glycol derivatives, sugars, ethoxylated and/or propoxylated esters and ethers, urea, sodium PCA, alcohols, ethanol, isopropyl alcohol, and combinations thereof. In one embodiment, the carrier is propylene glycol. Typically, the composition contains a carrier in an amount from about 1% by weight of the composition to about 99.9% by weight of the composition, more typically from about 2% by weight of the composition to about 95% by weight of the composition, and more typically from about 5% by weight of the composition to about 90% by weight of the composition.

Thermo-reversible gelling agents are defined as ingredients that are soluble, partially soluble, or miscible in a hydrophilic carrier at elevated temperatures, such as 50° C., wherein the agents have the ability to thicken the carrier when cooled to 25° C., but will be less viscous at 50° C. when application to a substrate is necessary. Suitable hydrophilic carriers include water, glycols, e.g., propylene glycol. Thermo-reversible gelling agents for use in the composition may include salts of fatty acids such as sodium stearate, sodium palmitate, potassium stearate. These salts can be added to the composition or can be created in-situ by addition of the fatty acid and neutralizing with appropriate base. An example of in-situ formation of the composition is to provide stearic acid and sodium hydroxide to produce sodium stearate. Other common thermo-reversible gelling agents could include, e.g., polyethylene glycols and derivatives such as PEG-20, PEG-150 distearate, PEG-150 pentaerythrityl tetrastearate, disteareth-75 IPDI, disteareth-100 IPDI, fatty alcohols, e.g., cetyl alcohol, fatty acids such as stearic acid, hydroxystearic acid and its derivatives, and combinations thereof.

In addition to the carrier and thermo-reversible gelling agent, the composition can contain various other ingredients and components. Examples of other ingredients that may be included within the composition are emollients, sterols or sterol derivatives, natural and synthetic fats or oils, viscosity enhancers, rheology modifiers, polyols, surfactants, alcohols, esters, silicones, clays, starch, cellulose, particulates, moisturizers, film formers, slip modifiers, surface modifiers, skin protectants, humectants, sunscreens, and the like.

Pharmaceutical Compositions and/or Preparations:

Embodiments described herein further relate to pharmaceutical compositions and/or preparations comprising one or more of the aforementioned pH responsive materials and a carrier. The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The pharmaceutical compositions may be prepared by any suitable means known in the art. Examples of such compositions include those adapted for: (a) topical application, e.g., articles (e.g., gauzes, pads, swabs, dressings), creams, ointments, gels, lotions, etc.; (b) parenteral administration, e.g., subcutaneous, intramuscular or intravenous injection as a sterile solution or suspension; (c) oral administration, external application (e.g. drenches including aqueous and non-aqueous solutions or suspensions), tablets, boluses, powders, granules, pellets for admixture with feedstuffs, pastes for application to the tongue, etc.

In certain embodiments, the pharmaceutical compositions may comprise one or more antibiotic agents. As used herein, the term “antibiotic” or “antimicrobial agent” refers to a substance that inhibits the growth of or destroys microorganisms. Preferably, the antibiotic is useful in curbing the virulence of an infectious agent and/or treating an infectious disease. Antibiotic also refers to semi-synthetic substances wherein a natural form produced by a microorganism, e.g., yeast or fungus is structurally modified.

In some embodiments, the antibiotic is selected from the group consisting of β-lactams (including, β-lactamase inhibitors and cephalosporins), fluoroquinolones, aminoglycosides, tetracyclines and/or glycylcyclines and/or polymyxins. Any combination of antimicrobial agents may also be employed, e.g., at least one β-lactam and at least one fluoroquinolone; at least one aminoglycoside and one cephalosporin; at least one β-lactam and one β-lactamase inhibitor, optionally together with an aminoglycoside, etc.

As used herein, the term “β-lactam” inhibitor includes natural and semi-synthetic penicillins and penicillin derivatives, e.g., benzathine penicillin, benzylpenicillin (penicillin G), phenoxymethylpenicillin (penicillin V), procaine penicillin and oxacillin; methicillin, dicloxacillin and flucloxacillin; temocillin; amoxicillin and ampicillin; azlocillin, carbenicillin, ticarcillin, mezlocillin and piperacillin; biapenem, doripenem, ertapenem, imipenem, meropenem, panipenem and PZ-601; cephalexin, cephalothin, cefazolin, cefaclor, cefuroxime, cefamandole, cefotetan, cefoxitin, cefotaxime, and cefpodoxime; cefepime and cefpirome; cefadroxil, cefixime, cefprozil, cephalexin, cephalothin, cefuroxime, cefamandole, cefepime and cefpirome; cefoxitin, cefotetan, cefmetazole and flomoxef; tigemonam, nocardicin A and tabtoxin; clavulanic acid, moxalactam and flomoxef. Fluoroquinolones include, ciprofloxacin, garenoxacin, gatifloxacin, gemifloxacin, levofloxacin, and moxifloxacin. Aminoglycosides include, for e.g., kanamycin, amikacin, tobramycin, dibekacin, gentamicin, sisomicin, netilmicin, neomycin B, neomycin C, neomycin E (paromomycin) and streptomycin, including, synthetic derivatives clarithromycin and azithromycin. Tetracyclines include naturally-occurring compounds (e.g., tetracycline, chlortetracycline, oxytetracycline, demeclocycline) or semi-synthetic agents (e.g., lymecycline, meclocycline, methacycline, minocycline, rolitetracycline). Glycylcyclines (e.g., minocycline/tigecycline) are derived from tetracyclines. Polymyxins include, e.g., polymyxin B and polymyxin E (colistin).

In certain embodiments, the compositions may contain an antibiotic at a concentration of 0.1 mg/mL, 0.5 mg/L, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL 44 mg/mL, 45 mg/mL, 50 mg/mL, 60 mg/mL, 70 mg/mL, 80 mg/m, 90 mg/mL, 100 mg/mL, 150 mg/mL, 200 mg/mL, 250 mg/mL, 300 mg/mL, 400 mg/mL, 500 mg/mL, or more. For example, imipenem and ertapenem may be used in the concentrations of 50, 30, 20, 15, 10, 5 and 1 mg/mL.

Wound Dressings:

Disclosed herein, in certain embodiments, are wound dressings comprising wound dressing materials as described herein, e.g., pH responsive materials. In some embodiments, the wound dressings consist essentially of the wound dressing materials as described herein, e.g., pH responsive materials.

In one embodiment, the wound dressing disclosed herein are biocompatible, biodegradable, non-immunogenic and readily commercially available.

In one embodiment, the pH responsive materials are provided in the form of particles, such as fiber particles or powder particles, optionally containing a medicament. In particular, the materials preferably contain PG fibers.

The compositions may preferably comprise an intimate mixture of the dressing material and other compounds. For instance, in one embodiment, the intimate mixture comprises a mixed solution or dispersion of the dressing material and a suitable vehicle, such as a solvent, or a solid composition produced by removing solvent from such a solution or dispersion. Under this embodiment, the dressing material makes up at least 5%, more preferably at least 10%, 20%, 30%, 50%, 75%, 90% or greater % by weight of the material. In certain preferred embodiments, the material consists essentially of the dressing material.

Other components of the material may include 0-25% by weight, for example from about 1 to about 20% by weight, of one or more other biocompatible polysaccharides, for example alginates such as sodium alginate or calcium alginate, starch derivatives such as sodium starch glycolate, cellulose derivatives such as methyl cellulose or carboxymethyl cellulose, or glycosaminoglycans such as hyaluronic acid or its salts, chondroitin sulfate or heparin sulfate. The materials may also comprise up to about 25% by weight, for example from about 1 to about 20% by weight, of one or more structural proteins selected from the group consisting of fibronectin, fibrin, laminin, elastin, collagen and mixtures thereof. In some embodiments, the protein comprises collagen, and in some embodiments, it consists essentially of collagen. The materials may also comprise up to about 20% by weight, preferably from about 2% to about 10% by weight of water. The materials may also contain 0-40% by weight, for example from about 5 to about 25% by weight, of a plasticizer, preferably a polyhydric alcohol such as glycerol or sorbitol.

In certain embodiments, the materials may also comprise up to about 10% by weight, for example from about 0.01 to about 5% by weight, typically from about 0.1 to about 2% by weight of one or more therapeutic wound healing agents, such as non-steroidal anti-inflammatory drugs (e.g., acetaminophen), steroids, local anesthetics, antimicrobial agents, or growth factors (e.g., fibroblast growth factor or platelet derived growth factor). The antimicrobial agent may, for example, comprise an antiseptic, an antibiotic, or mixtures thereof. Preferred antibiotics include tetracycline, penicillins, terramycins, erythromycin, bacitracin, neomycin, polymycin B, mupirocin, clindamycin and mixtures thereof. Preferred antiseptics include silver, including colloidal silver, silver salts including salts of one or more of the anionic polymers making up the material, silver sulfadiazine, chlorhexidine, povidone iodine, triclosan, sucralfate, quaternary ammonium salts and mixtures thereof. These medicated wound dressing materials according to the disclosed technology provide sustained release of the therapeutic agents as the wound dressing material breaks down in use.

All of the above percentages are on a dry weight basis. Preferably, the weight ratio of the wound dressing material to other auxiliary agents and materials is from about 1:99 to about 99:1. More preferably, the weight ratio is in the range about 1:9 to about 9:1, more preferably it is in the range about 4:1 to about 1:4, still more preferably in the range about 2:1 to about 1:2.

The material may be in any convenient form, such as a powder, microspheres, flakes, a mat or a film.

In certain embodiments, the material is in the form of a semisolid or gel ointment for topical application.

In certain embodiments, the material is in the form of a freeze-dried or solvent-dried bioabsorbable sponge for application to a chronic wound. Preferably, the average pore size of the sponge is in the region of 10-500 μm, more preferably about 100-300 μm. A suitable sponge has been made by freeze-drying or solvent drying an aqueous dispersion comprising pH responsive materials as described herein, together with suitable therapeutic agents.

In yet other embodiments, the material is in the form of a flexible film, which may be continuous or interrupted (e.g. perforated). The flexible film preferably comprises a plasticizer to render it flexible, such as glycerol.

The ready availability of both gel forming polymers, e.g., cellulose derivatives, having a range of controllable properties means that the properties of the compositions the disclosed technology can be controlled to an exceptional degree. In particular, the rate of biological absorption, porosity and density of the materials can be controlled.

In one embodiment, provided herein are wound dressing materials in sheet form, comprising an active layer of a composition comprising the pH responsive materials disclosed herein. The active layer would normally be the wound contacting layer in use, but in some embodiments, it could be separated from the wound by a liquid-permeable top sheet. In one embodiment, the area of the active layer is from about 1 cm² to about 400 cm², particularly from about 4 cm² to about 100 cm².

In another embodiment, the wound dressing material further comprises a backing sheet extending over the active layer opposite to the wound facing side of the active layer. Preferably, the backing sheet is larger than the active layer such that a marginal region of width 1 mm to 50 mm, preferably 5 mm to 20 mm extends around the active layer to form a so-called island dressing. In such cases, the backing sheet is preferably coated with a pressure sensitive medical grade adhesive in at least its marginal region.

In embodiments wherein the dressing material comprises a backing sheet, the back sheet is substantially liquid-impermeable. In another embodiment, the backing sheet is semipermeable, e.g., the backing sheet is preferably permeable to water vapor, but not permeable to liquid water or wound exudate. Preferably, the backing sheet is also microorganism-impermeable. Suitable continuous conformable backing sheets will preferably have a moisture vapor transmission rate (MVTR) of the backing sheet alone of 300 to 5000 g/m²/24 hrs, preferably 500 to 2000 g/m²/24 hrs at 37.5° C. at 100% to 10% relative humidity difference. The backing sheet thickness is preferably in the range of 10 to 1000 micrometers, more preferably 100 to 500 micrometers.

Suitable polymers for forming the backing sheet include polyurethanes and poly alkoxyalkyl acrylates and methacrylates. Preferably, the backing sheet comprises a continuous layer of a high density blocked polyurethane foam that is predominantly closed-cell. A suitable backing sheet material is a polyurethane film.

In wound dressings comprising a backing layer comprising an adhesive, the adhesive layer should be moisture vapor transmitting and/or patterned to allow passage of water vapor. The adhesive layer is preferably a continuous moisture vapor transmitting, pressure-sensitive adhesive layer of the type conventionally used for island-type wound dressings, for example, a pressure sensitive adhesive based on acrylate ester copolymers, polyvinyl ethyl ether and polyurethane. Polyurethane-based pressure sensitive adhesives may be selectively used.

In another embodiment, the dressing may comprise further layers of a multilayer absorbent article may be built up between the active layer and the protective sheet. For example, these layers may comprise an apertured plastic film to provide support for the active layer in use, in which case the apertures in the film are preferably aligned in register with the apertures in the hydrogel layer.

Still further, in other embodiments, the dressing may comprise an absorbent layer between the active layer and the protective sheet, especially if the dressing is for use on exuding wounds. The optional absorbent layer may be any of the layers conventionally used for absorbing wound fluids, serum or blood in the wound healing art, including gauzes, nonwoven fabrics, superabsorbents, hydrogels and mixtures thereof. Preferably, the absorbent layer comprises a layer of absorbent foam, such as an open celled hydrophilic polyurethane foam. In other embodiments, the absorbent layer may be a nonwoven fibrous web, for example a carded web of viscose staple fibers.

In certain embodiments, the wound dressing may be protected by a removable cover sheet. The cover sheet is normally formed from flexible thermoplastic material. Suitable materials include polyesters and polyolefins. Preferably, the adhesive-facing surface of the cover sheet is a release surface. That is to say, a surface that is only weakly adherent to the active layer and the adhesive on the backing sheet to assist peeling of the hydrogel layer from the cover sheet. For example, the cover sheet may be formed from a non-adherent plastic such as a fluoropolymer, or it may be provided with a release coating such as a silicone or fluoropolymer release coating.

In one embodiment, the wound dressing is sterile and packaged in a microorganism-impermeable container.

Kits:

In certain embodiments, the disclosed technology provides kits comprising, in one or separate compartments, the pH responsive materials described herein, optionally together with an excipient, carrier or oil. The kits may further comprise additional ingredients, e.g., gelling agents, emollients, surfactants, humectants, viscosity enhancers, emulsifiers, etc., in one or more compartments. The kits may optionally comprise instructions for formulating an article for diagnosing, detecting or treating wounds, e.g., chronic or infected wounds. The kits may also comprise instructions for using the components, either individually or together, in the treatment of wounds.

In a related embodiment, the disclosed technology provides kits comprising a package and at least one absorbent article (described above) comprising the aforementioned compositions. Alternately, the kits may comprise the individual components separately, optionally together with secondary information, useable in or with the package.

Other embodiments disclosed herein relate to the use of the composition for the preparation of a dressing for the treatment of a wound. Preferably, the wound is a chronic wound, for example a wound selected from the group consisting of venous ulcers, decubitis ulcers and diabetic ulcers.

Systems:

Embodiments of the disclosed technology further provide for diagnostic systems comprising the aforementioned compositions and/or kits.

The various components of the diagnostic systems may be provided in a variety of forms. For example, the pH responsive materials described herein may be provided as a lyophilized reagent. These lyophilized reagents may be pre-mixed before lyophilization so that when reconstituted they form a complete mixture with the proper ratio of each of the components ready for use in the assay. In addition, the diagnostic systems of the disclosed technology may contain a reconstitution reagent for reconstituting the lyophilized reagents of the kit.

Methods of Making pH Responsive Materials

In some embodiments, as described herein, are methods of making pH responsive materials. In certain embodiments, the method of making a pH responsive material comprises the steps of dissolving a linking moiety in an acidic solution, immersing an anchor material in the acidic solution, drying the anchor material, and soaking the anchor material in a solution comprising a pH indicator.

In certain embodiments, the method comprises dissolving a linking moiety in an acidic solution. In certain embodiments, the acidic solution is an aqueous solution. In certain embodiments, the acidic solution comprises an organic solvent. In certain embodiments, the acidic solution comprises an inorganic acid. In certain embodiments, the inorganic acid is selected from hydrochloric acid, sulfuric acid, phosphoric acid, hydrobromic acid, nitric acid, or combinations thereof. In certain embodiments, the acidic solution comprises an organic acid. In certain embodiments, the organic acid is selected from formic acid, acetic acid, trifluoroacetic acid, trichloroacitic acid, or combinations thereof.

In certain embodiments, the acidic solution has an acid concentration of up to about 1, 5, 10, 20, 30, 40, 50, 60, 70, 80, 90, 100, 1000 μM. In certain embodiments, the acid concentration is at most about 1 μM. In certain embodiments, the acid concentration is at most about 5 μM. In certain embodiments, the acid concentration is at most about 10 μM. In certain embodiments, the acid concentration is at most about 20 μM. In certain embodiments, the acid concentration is at most about 30 μM. In certain embodiments, the acid concentration is at most about 1 μM. In certain embodiments, the acid concentration is at most about 40 μM. In certain embodiments, the acid concentration is at most about 50 μM. In certain embodiments, the acid concentration is at most about 60 μM. In certain embodiments, the acid concentration is at most about 80 μM. In certain embodiments, the acid concentration is at most about 70 μM. In certain embodiments, the acid concentration is at most about 90 μM. In certain embodiments, the acid concentration is at most about 100 μM. In certain embodiments, the acid concentration is at most about 1000 μM. In certain embodiments, the acid concentration is about 57 μM.

In certain embodiments, the linking moiety is a silane reagent. In certain embodiments, the silane reagent comprises an epoxide functional group. In certain embodiments, the silane reagent is selected from GPTMS, Diethoxy(3-glycidyloxypropyl)methylsilane, 3-Glycidoxypropyldimethoxymethylsilane, epoxycyclohexyl)ethyltriethoxysilane, 2-(3,4-epoxycyclohexyl)ethyltrimethoxysilane, or combinations thereof. In certain embodiments, the silane reagent is GPTMS. In certain embodiments, the silane reagent is Diethoxy(3-glycidyloxypropyl)methylsilane. In certain embodiments, the silane reagent is 3-Glycidoxypropyldimethoxymethylsilane. In certain embodiments, the silane reagent is 2-(3,4-epoxycyclohexyl)ethyltriethoxysilane. In certain embodiments, the silane reagent is 2-(3,4-epoxycyclohexyl)ethyltrimethoxysilane.

In certain embodiments, an anchor material is immersed in the acidic solution. In certain embodiments, the anchor material comprises OH-bearing surfaces. In certain embodiment, the anchor material comprises a polysaccharide, a cellulose, or a monomer thereof, an oligomer thereof, a derivative thereof, a mixture or a combination thereof. In certain embodiments, the anchor material comprises a polysaccharide selected from hydroxypropyl methylcellulose (HPMC), hydroxyethyl cellulose, hydroxymethyl cellulose, D-galactopyranoside, or a derivative thereof. In certain embodiments, the anchor material is a solid support. In certain embodiments, the solid support is selected from cotton, paper, filter paper, a cotton swab, a wound dressing, or a cellulose particle. In certain embodiments, the solid support is a cellulose particle. In certain embodiments, the cellulose particle has a particle size of up to about 1, 2, 3, 4, 5, 6, 7, 8, 9, 10, 15, 20, 25, 30, 35, 40, 45, 50, 60, 70, 80, 90, or 100 μm. In certain embodiments, the cellulose particle has a particle size of about 20 μm. In certain embodiments, the solid support is a cotton swab. In certain embodiments, the cotton swab is suitable for using in a wound.

In certain embodiments, the anchor material is immersed in the acidic solution for at least about 5, 10, 15, 20, 25, 30, 35, 40, 45, 50, 55, or 60 minutes. In certain embodiments, the anchor material is immersed in the acidic solution for at least about 5 minutes. In certain embodiments, the anchor material is immersed in the acidic solution for at least about 10 minutes. In certain embodiments, the anchor material is immersed in the acidic solution for at least about 15 minutes. In certain embodiments, the anchor material is immersed in the acidic solution for at least about 20 minutes. In certain embodiments, the anchor material is immersed in the acidic solution for at least about 25 minutes. In certain embodiments, the anchor material is immersed in the acidic solution for at least about 30 minutes. In certain embodiments, the anchor material is immersed in the acidic solution for at least about 35 minutes. In certain embodiments, the anchor material is immersed in the acidic solution for at least about 40 minutes. In certain embodiments, the anchor material is immersed in the acidic solution for at least about 45 minutes. In certain embodiments, the anchor material is immersed in the acidic solution for at least about 50 minutes. In certain embodiments, the anchor material is immersed in the acidic solution for at least about 55 minutes. In certain embodiments, the anchor material is immersed in the acidic solution for at least about 60 minutes.

In certain embodiments, the acidic solution is at a temperature of at least about 60, 70, 80, 90, 100, 110, or 120° C. In certain embodiments, the acidic solution is at a temperature of at least about 60° C. In certain embodiments, the acidic solution is at a temperature of at least about 70° C. In certain embodiments, the acidic solution is at a temperature of at least about 80° C. In certain embodiments, the acidic solution is at a temperature of at least about 90° C. In certain embodiments, the acidic solution is at a temperature of at least about 100° C. In certain embodiments, the acidic solution is at a temperature of at least about 110° C. In certain embodiments, the acidic solution is at a temperature of at least about 120° C.

In certain embodiments, the method of making a pH responsive material comprises a drying step after immersing the anchor material in an acidic solution with a linking moiety. In certain embodiments, the drying step is carried out with a heat-gun. In certain embodiments, the drying step heats the anchor material to a surface temperature of up to about 75° C.

In certain embodiments, the method of making a pH responsive material comprises soaking the anchor material in a solution comprising a pH responsive element. In certain embodiments, the pH responsive element is at a concentration of 1 mg/mL in the solution.

In certain embodiments, the pH responsive element is a pH indicator. In certain embodiments, the pH indicator comprises multiple phenol functional groups. In certain embodiments, the pH indicator is selected from thymol blue, bromophenol blue, bromocresol green, bromocresol purple, bromothymol blue, phenol red, napththolpthalein, cresol red, cresolpthalein, phenolphthalein, or thymolpthalein. In certain embodiments, the pH indicator is bromocresol purple. In certain embodiments, the pH indicator is functionalized onto the linking moiety through a reaction of a phenol functional group of the pH indicator with an epoxide function group of the linking moiety.

In certain embodiments, the method of making a pH responsive material further comprises a washing step. In certain embodiments, the method of making a pH responsive material comprises the steps of dissolving a linking moiety in an acidic solution, immersing an anchor material in the acidic solution, drying the anchor material, soaking the anchor material in a solution comprising a pH indicator, and washing the anchor material. In certain embodiments, the anchor material is washed with a buffer. In certain embodiments, the anchor material is washed with a phosphate buffer. In certain embodiments, the phosphate buffer has a concentration of 10 mM. In certain embodiments, the phosphate buffer has a pH of 3.0.

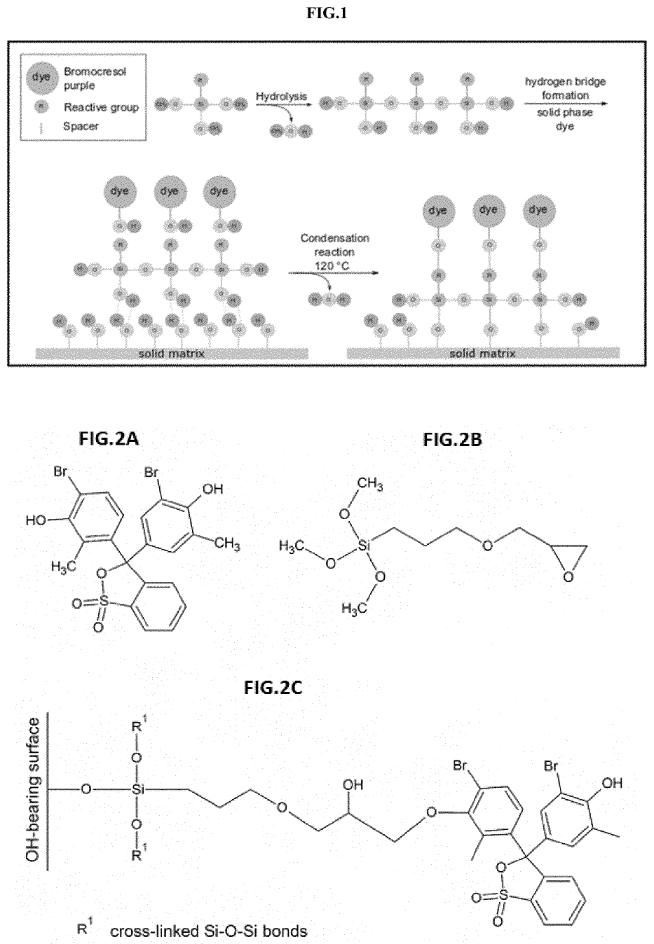

A non-limiting, proposed possible reaction mechanism scheme for a method of making the pH responsive materials is shown in FIG. 1. The top of FIG. 1 shows a partial hydrolysis of a silane monomer, and subsequent polymerization into a siloxane. FIG. 1 then shows hydrogen bond formation between an OH-bearing surface (“solid matrix” in the figure) and the siloxane polymer. FIG. 1 then illustrates a proposed, non-limiting, condensation reaction, resulting in bonds being formed between the siloxane and the OH-bearing surface, as well as between the siloxane and a dye molecule. FIG. 2A shows the structure of bromocresol purple. FIG. 2B shows the structure of GPTMS. FIG. 2C shows a non-limiting, proposed structure of the resulting pH responsive material made from an OH-bearing surface, GPTMS, and bromocresol purple.

Medical and Diagnostic Devices

In certain embodiments, the pH responsive materials described herein are incorporated into a medical or diagnostic device. Non-limiting examples of medical devices include dressings, wound dressings, swabs, and bandages. In certain embodiments, the pH responsive material is incorporated into a medical or diagnostic device by spraying, printing, or depositing the cellulose particle. In certain embodiments, the pH responsive material is sprayed, printed, or deposited on a solid phase of the medical or diagnostic device. In certain embodiments, the solid phase of the medical or diagnostic device comprises a dressing, a wound dressing, a bandage, filter paper, or a test strip.

In certain embodiments, the pH responsive material incorporated into a medical or diagnostic device comprise cellulose particles. In certain embodiments, the cellulose particle is incorporated into a medical or diagnostic device by spraying, printing, or depositing the cellulose particle. In certain embodiments, the cellulose particle is sprayed, printed, or deposited on a solid phase of the medical or diagnostic device. In certain embodiments, the solid phase of the medical or diagnostic device comprises a dressing, a wound dressing, a bandage, filter paper, or a test strip.

Other embodiments include pH responsive materials described herein printed on dressing or solid support materials, dipstick devices with indicator disks arranged in various arrays, and devices with separate sample preparation chamber that transfer a sample of a bodily fluid or wound fluid to a standalone diagnostic device that uses reagent pills, solutions, or disks in reaction chambers for detecting biomarkers associated with microbial detection. In further embodiments, indicator reagents are printed, sprayed, or overlayed on support materials, such as dressing, wound dressing, bandage, filter paper, and test strips.

Diagnostic and Therapeutic Methods:

In one embodiment, the compositions, dressing materials, articles, kits and systems described herein are useful in diagnosing or treating wounds, particularly chronic or infected wounds. Although any type of wound may be diagnosed and/or treated, the embodiments are particularly suitable for diagnosing and treating wounds that exude wound fluid. For example, the wound may be a chronic or acute wound. Representative examples of chronic wounds include, e.g., venous ulcers, pressure sores, decubitis ulcers, diabetic ulcers and chronic ulcers of unknown aetiology. Representative examples of acute wounds include, e.g., acute traumatic laceration, perhaps resulting from an intentional operative incision.

As used herein, the term “a wound fluid” refers to any wound exudate or other fluid (suitably substantially not including blood) that is present at the surface of the wound, or that is removed from the wound surface by aspiration, absorption or washing. The determining, measuring or quantifying is suitably carried out on wound fluid that has been removed from the body of the patient, but can also be performed on wound fluid in situ. The term “wound fluid” does not normally refer to blood or tissue plasma remote from the wound site. The wound fluid is mammalian wound fluid, suitably human wound fluid.

In one embodiment, the diagnostic method comprises contacting a wound with at least one composition comprising a pH responsive material as described herein, a dressing material comprising such pH responsive material, an article comprising such pH responsive materials or compounds, kits comprising such materials or compounds, or a system comprising such materials or compounds described herein; and measuring a parameter associated with the wound. In a specific embodiment, the parameter being measured is pH.

In the aforementioned embodiments, the measurement may be made either in situ or ex situ. As used herein, the term “in situ” refers to processes, events, objects, or components that are present or take place within the context of the system or device, including, the surrounding environment, for example, the biological material with which the composition, article, system or device is in contact with. As an example, an in situ reaction may refer to the reaction of the various components present in the device (e.g., a pH responsive material as described herein), including, components provided by the human skin tissue (e.g., wound exudate containing the enzyme). The term is contrasted with ex situ, which refers to outside of the environment.

In a second embodiment, the measurement is performed ex situ, e.g., removing the fluid from the wound for analysis in the apparatus or device of the disclosed technology.

Suitably, the measurement is made in situ.

Embodiments disclosed herein further relate to treatment of chronic or infected wounds using the compositions, materials, articles, dressings, kits and/or systems described herein. The therapeutic embodiment includes, contacting a composition, material, article, dressing, kit, system or devices of the disclosed technology with a subject in need thereof. Optionally, the method may include determination of whether the subject is responding to the treatment.

The skilled person would be able to easily identify whether wounds are “responsive to treatment” or not. In particular, the skilled person will readily be able to determine the levels of the proteases identified in the present claims that are predictive or indicative of a good response or poor response to wound treatment, particularly to treatment with wound dressings comprising oxidized cellulose. The terms “responsive” and “responder(s)” as used herein refer to wounds that are considered to respond well to wound treatment, particularly to treatment with a pharmacological agent, e.g., antibiotics. Similarly, “non-responsive” and “non-responder(s)” refers to wounds that are not considered to respond well to wound treatment, particularly to treatment with the pharmacological agent, e.g., antibiotics. For instance, patients who exhibit better than 50% wound closure after 4 weeks of wound treatment are considered to be responsive to said treatment.

In certain embodiments, a patient may be simultaneously diagnosed and treated with the compositions, articles, systems, or devices described herein. When used herein, the term “simultaneously” means performing the stated objectives, e.g., diagnosis and treatment, together.

In certain embodiments, a patient may be sequentially diagnosed and treated with the compositions, articles, systems, or devices described herein. When used herein, the term “sequentially” means the stated objectives, e.g., diagnosis and treatment, are temporally or spatially separated, e.g., diagnosis prior to treatment or diagnosis following treatment or a combination thereof, e.g., 1^{st} diagnosis→treatment==>2^{nd} diagnosis.

Embodiments described herein further enable a care giver or a patient to determine quickly and reliably whether a wound is likely to be non-healing, and to select an appropriate therapy based on this determination. For example, non-healing wounds may require the application of special wound dressings such as wound dressings comprising specific therapeutic agents, to promote healing. Accordingly, embodiments described herein further provide methods of treatment of a wound, e.g., chronic or infected wounds, comprising determining whether a wound is healing or non-healing, followed by applying a wound dressing comprising a therapeutic agent to the wound if it is non-healing.

Embodiments described herein provide methods and assays for diagnosis or detection of infected wounds. The methods are suitable for the detection of bacterial infectious agents. In one embodiment, the wounds are infected with gram-negative bacteria. Typical gram-negative bacteria include proteobacteria such as E. coli, Salmonella, Pseudomonas, and Helicobacter, and cyanobacteria. When classified in connection with medicine, they include Pseudomonas aeruginosa and Hemophilus influenzae causing the disturbance of the respiratory system, Escherichia coli and Proteus mirabilis causing the disturbance of the urinary system, and Helicobacter pylori and Bacillus gaertner causing the disturbance of the alimentary system and micrococci such as Neisseria meningitidis, Moraxella catarrhalis, and Neisseria gonorrhea.

In another embodiment, the wounds are infected with gram-positive bacteria. By “gram-positive bacteria” is meant a bacterium or bacteria that contain(s) teichoic acid (e.g., lipoteichoic acid and/or wall teichoic acid), or a functionally equivalent glycopolymer (e.g., a rhamnopolysaccharide, teichuronic acid, arabinogalactan, lipomannan, and lipoarabinomannan) in its cell wall. Non-limiting examples of functionally equivalent glycopolymers are described in Weidenmaier et al., Nature, 6:276-287, 2008.

The bacteria include pathogenic bacteria that infect mammalian hosts (e.g., bovine, murine, equine, primate, feline, canine, and human hosts). Examples of such pathogenic bacteria include, e.g., members of a bacterial species such as Bacteroides, Clostridium, Streptococcus, Staphylococcus, Pseudomonas, Haemophilus, Legionella, Mycobacterium, Escherichia, Salmonella, Shigella, Vibrio, or Listeria. Some clinically relevant examples of pathogenic bacteria that cause disease in a human host include, but are not limited to, Bacillus anthraces, Bacillus cereus, Bordetella pertussis, Borrelia burgdorferi, Brucella aborus, Brucella canis, Brucella melitensis, Brucella suis, Campylobacter jejuni, Chlamydia pneumoniae, Chlamydia psittaci, Chlamydia trachomatis, Clostridium botulinum, Clostridium difficile, Clostridium perfringens, Clostridium tetani, Corynebacterium diphtheriae, Enterococcus faecalis, vancomycin-resistant Enterococcus faecalis, Enterococcus faecium, Escherichia coli, enterotoxigenic Escherichia coli (ETEC), enteropathogenic Escherichia coli, E. coli O157:H7, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Proteus, Pseudomonas aeruginosa, Rickettsia rickettsia, Salmonella typhi, Salmonella typhimurium, Shigella sonnei, Staphylococcus aureus, Staphylococcus epidermis, Staphylococcus saprophyticus, methicillin-resistant Staphylococcus aureus (MRSA), vancomycin-resistant Staphylococcus aureus (VSA), Streptococcus agalactiae, Streptococcus pneumoniae, Streptococcus pyogenes, Treponema pallidum, Vibrio cholerae, and Yersinia pestis.

In some embodiments, the infectious bacteria is selected from the group consisting of Clostridium difficile, Carbapenem-Resistant Enterobacteriaceae (CR-Klebsiella spp; CR-E. coli), and Neisseria gonorrhoeae. In another embodiment, the infectious bacteria is selected from the group consisting of multidrug-resistant Acinetobacter, drug-resistant Campylobacter, extended spectrum β-Lactamase (ESBL)-producing enterobacteriaceae, vancomycin-resistant enterococcus, multidrug-resistant Pseudomonas aeruginosa, drug-resistant non-typhoidal Salmonella, drug-resistant Salmonella enterica serovar Typhi, drug-resistant Shigella, methicillin-resistant Staphylococcus aureus (MRSA), drug-resistant Streptococcus pneumoniae, and drug-resistant Tuberculosis. In another embodiment, the infectious bacteria is selected from the group consisting of vancomycin-resistant Staphylococcus aureus, erythromycin-resistant Group A Streptococcus, clindamycin-Resistant Group B Streptococcus.

In certain embodiments, the chronic or infected wounds are found in host subjects. Preferably, the hosts are mammals, e.g., a rodent, a human, a livestock animal, a companion animal, or a non-domesticated or wild animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoo animal. As used herein, a “zoo animal” refers to an animal that may be found in a zoo. Such animals may include non-human primates, large cats, wolves, and bears. In an exemplary embodiment, the subject is a human.

In one aspect, provided herein are methods of diagnosing a status of a wound, comprising the steps of: contacting the wound or wound fluid with a pH responsive material, and detecting a color change of the pH responsive material. In another aspect, provided herein are methods of treating an infected wound comprising the steps of: contacting the wound or a wound fluid with a pH responsive material; detecting a color change of the pH responsive material; and administering a treatment for an infection.

Preferably, the diagnosis and treatment is conducted in situ. Embodiments described herein therefore allow diagnosis and treatment of wounds in an easy, non-invasive manner. For instance, the diagnosis may be made in real time and the treatment may be applied to the infected wound or to the patient (systemically) and the progress of wound treatment be monitored over real-time, e.g., a decrease in wound pH due to wound healing.

In another aspect, provided herein are methods of detecting infection in an airway, comprising contacting the pH responsive materials with the fluid from the infected organ either via specific sampling or via long-term contact with a ventilation device.

### Examples

The structures, materials, compositions, and methods described herein are intended to be representative examples, and it will be understood that the scope of the disclosure is not limited by the scope of the examples. Those skilled in the art will recognize that the embodiments and disclosed technology may be practiced with variations on the disclosed structures, materials, compositions and methods, and such variations are regarded as within the ambit of the disclosure.

All ionic compounds were handled and isolated as salts with various counter ions, depending on the last step and not further specified.

Bromocresol purple (BCP), acetic acid (AA), (3-glycidyloxypropyl) trimethoxysilane (GPTMS), phosphoric acid, citric acid, disodium hydrogen phosphate, sodium dihydrogen phosphate, sodium carbonate, sodium hydroxide, sodium chloride and hydrochloric acid were all of analytical grade, obtained from Sigma-Aldrich (St. Louis, USA) and used as received. The cellulose support materials (Whatman filter paper grade 1 and Sigmacell cellulose Type 20, 20 μm) were also obtained from Sigma-Aldrich. The cotton swabs (EH11.1) were obtained from Carl Roth GmbH & Co. KG (Karlsruhe, Germany). The dressing (Aquacel® Foam, Non-Adhesive, 10 cm×10 cm) was obtained from ConvaTec (Deeside, UK). The sterile wound swabs (FLOQSwabs, Regular, Sterile Single Wrapped, Molded bp 100 mm) were obtained from Copan (Brescia, Italy).

### Example 1. Functionalization of Filter Paper and Cotton Swabs

89.0 ml 57 μM AA was stirred with a magnetic stirrer and 11.0 ml of GPTMS was added dropwise. The reaction solution was stirred for additional 15 min until no turbidity was observable. Whatman filter paper and the cotton swabs were fully soaked in the reaction solution and dried with a heat-gun (generating a surface temperature of 75° C. on the paper) for a homogenous distribution on the surface. The dry GPTMS-modified material was then soaked in a 1 mg/ml BCP solution (in 57 μM acetic acid) and pre-dried with a heat-gun. The condensation reaction was carried out in a compartment drier at 120° C. for 20 minutes. Excess dye was removed from the functionalized BCP (FBCP) by several washing steps with phosphate buffer (pH 3.0, 10 mM, total volume 2 L). Dye removed in the eluate was monitored by spectrophotometer and washing continued until no dye could be detected after incubation in buffer for 60 minutes.

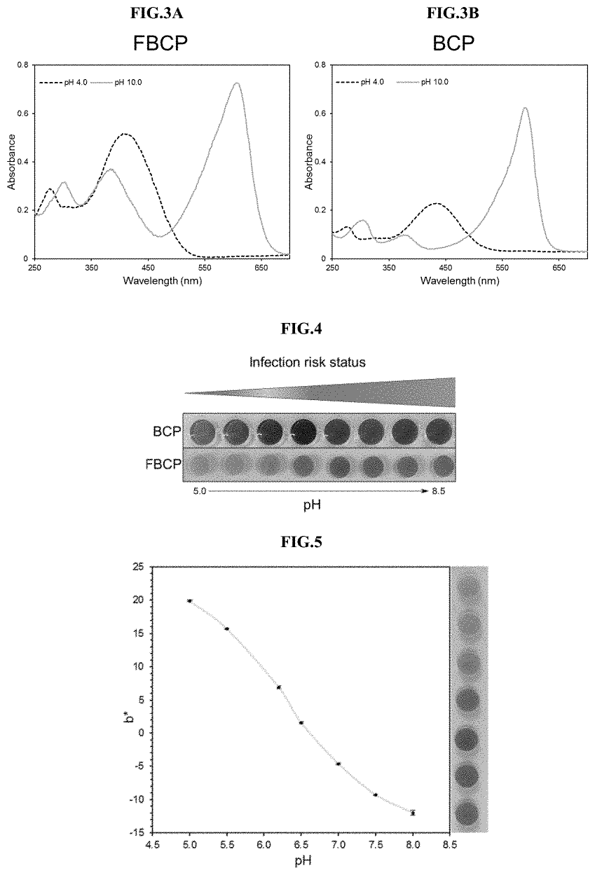

The functionalized filter paper was then characterized with via UV-Vis analysis and compared with non-functionalized BCP. (FIG. 3). Absorbance scans were performed at acidic and alkaline pH. The background of the cellulose layer from the filter paper was subtracted from the FBCP scans to give better comparable figures. Both variants of the dye show a clear pH-dependent color change. Nevertheless, upon immobilization a shift of the absorbance maximum could be observed in both the acidic and the alkaline form. Whereas a shift from 432 nm to 406 nm was observed for the acidic form, a shift from 588 nm to 606 nm was recorded for the alkaline form. Additionally, the new indicator material shows a clear peak at 384 nm compared to the unbound pH indicator, which explains the blue shift of the new material under alkaline conditions. This additional peak stretches the usable pH range to a wider area as shown in FIG. 4.

One aspect of the pH-responsive materials described in this example is simple readability of the color response by the naked eye. This allows for simple inspection and evaluation, and is optionally performed by choosing a color change that gives contrast to likely colors present in wound exudate. A yellow to blue shift from regular to elevated pH values as infection risk increases is one option. The color change of the functionalized filter paper was quantified using the CIELab color space concept which is based on the measurement of three values: L*, a* and b*. The L* value in this concept represents the brightness, the a* value the color change from green to red, and the b* value represents the color shift from yellow to blue. One clinically relevant pH range for the detection of infection in wounds is between the pH-values of 5.0 and 8.0. Hence, the b* value was used to quantify the color change of the functionalized material in this range. FIG. 5 shows the relationship between apparent color and the corresponding pH indicating that pH values between 5.0 and 8.0 can be distinguished with this material and give measurable colors from yellow to blue.

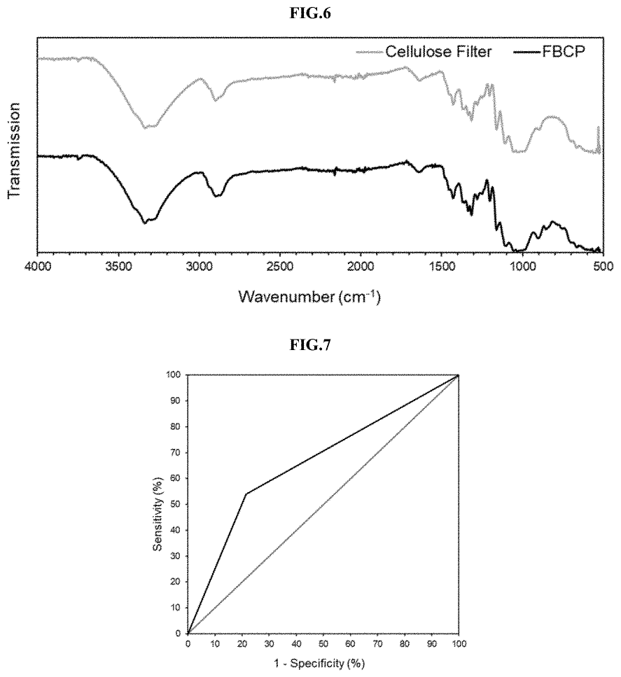

FTIR spectra recordings from the blank filter paper (cellulose filter) and the FBCP are provided in FIG. 6. Differences were detected in the area between 1200-700 cm⁻¹. The similarities in the spectra may be due to the relatively low concentration of BCP compared with the remaining components.

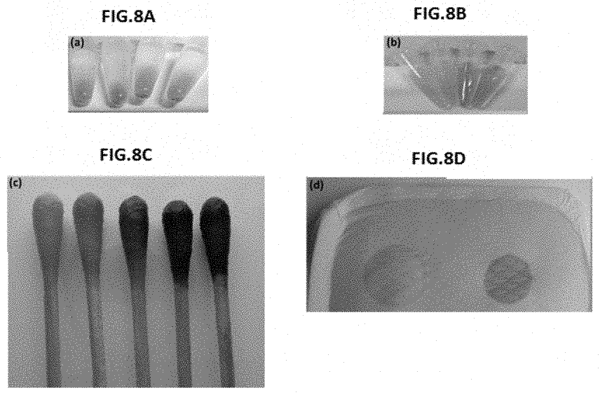

FIG. 8C shows the functionalized cotton swabs prepared as indicated above before and after contacting the swabs with various pH buffer (left to right=initial color, pH 5.0, 6.0, 7.0, 8.0).

### Example 2. Functionalization of Cellulose Particles

18.6 mg of cellulose particles (20 μm) were suspended in 100 μl of a 10% (v/v) GPTMS solution in 57 μM AA and incubated in a compartment drier at 120° C. for 20 minutes. The incubation step was carried out in an open glass vial. The dried functionalized particles were transferred into a new glass vial containing 100 μl of a BCP solution (1 mg/ml in 57 μM AA). The second reaction step was also carried out at 120° C. for 20 minutes in a compartment drier. The stained particles were re-suspended and washed in phosphate buffer (pH 3.0, 10 mM) until no dye could be detected in the supernatant.

Example uses of functionalized cellulose particles are shown in FIG. 8A and FIG. 8B. FIG. 8A shows settled particles after centrifugation of a suspension of the particles in various pH buffers (left to right=pH 5.0, 6.0, 7.0, 8.0). FIG. 8B shows particles in suspension in various pH buffers (left to right=pH 5.0, 6.0, 7.0, 8.0).

### Example 3. Integration into a Wound Dressing

A single layer of FBCP filter paper was inserted between the polyurethane layer and the top opaque adhesive layer of a non-adhesive Aquacel® Foam dressing. 0.5 ml of pH 5.0 and 8.0 buffer was pipetted onto the bottom hydrofiber layer of the dressing, respectively. After 5 min, the buffer reached the FBCP layer and color change documented by photography. The results can be seen in FIG. 8D

### Example 4. Diagnosis of Wound Infection

Wound fluid samples were obtained from 156 patients with open chronic wounds suitable for sampling via wound swab. The study was conducted at the departments of vascular surgery of Medisch Spectrum Twente hospital (Enschede), Ziekenhuisgroep Twente (Almelo), Streekziekenhuis Koningin Beatrix (Winterswijk), St. Jansdal hospital (Harderwijk), and at Livio homecare (region of Twente) and with informed consent of the patients and following approval from the Medical Ethical Committee Twente. Non-eligible patients (Under-age persons or the mentally incompetent) and patients without informed consent were excluded. Wounds were evaluated by clinicians based on the clinical appearance of the wounds and were classified into two groups: “infected” and “non-infected”. Regular sterile swabs were used for the sampling of 156 wounds and after the sampling, all swabs were suspended in 1 ml of unbuffered physiological saline solution. All wound fluids were analyzed by applying five μl of sample onto the FBCP filter paper. The occurrence of a blue color corresponding to pH values above 7.0 was used to classify the samples as positive. The pH results were compared with the clinicians' opinion on the infection status of the wound for the calculation of sensitivity and specificity of pH values above 7.0.

Following the measurement of pH of 156 wound fluid samples using our pH-sensing material 114 wounds had a pH <7.0 (green color, considered as non-infected) and 42 wounds had a pH ≥7.0 (blue color, considered infected). The sensitivity and specificity of the pH measurements to determine wound infection status, were calculated by comparing with wound status determined by clinical observation. The sensitivity was plotted against 1-specificity resulting in a receiver operator characteristic curve (FIG. 7) to visualize the diagnostic properties of the sensing material.

More than 50% of infected-classified wounds (Sensitivity 53.8%) showed an elevated pH and were detectable with the pH responsive material. This leads to a positive predictive value (PPV) of 33.3% (Table 1). The specificity was 78.5% with a false positive rate of only 21.5% and a negative predictive value (NPV) of 89.5%.

TABLE 1

| | | Clinical judgment | | | | |
|---|---|---|---|---|---|---|
| | | | Not | | | |
| | 156 | Infected | infected | PPV | NPV | AUC |
| Elevated | Infected | TP 14 | FP 28 | 0.33 | 0.89 | 0.66 |
| pH | (pH ≥ 7.0) | | | | | |
| | Not infected | FN 12 | TN 102 | | | |
| | (pH < 7.0) | | | | | |

While preferred embodiments of the disclosed technology have been shown and described herein, it will be obvious to those skilled in the art that such embodiments are provided by way of example only. Numerous variations, changes, and substitutions will now occur to those skilled in the art without departing from the disclosed technology. It should be understood that various alternatives to the embodiments of the disclosed technology described herein may be employed in practicing the disclosed technology. It is intended that the following claims define the scope of the disclosed technology and that methods and structures within the scope of these claims and their equivalents be covered thereby.

We claim:

1. A pH responsive material, comprising;
an anchor material;
a linking moiety; and
a pH responsive element.

2. The pH responsive material of claim 1, wherein the linking moiety connects the anchor material and the pH responsive element via covalent bonds.

3. The pH responsive material of claim 1, wherein the anchor material comprises OH-bearing surfaces.

4. The pH responsive material of claim 1, wherein the anchor material comprises a polysaccharide, a cellulose, or a monomer thereof, an oligomer thereof, a derivative thereof, a mixture or a combination thereof.

5. The pH responsive material of claim 1, wherein the anchor material comprises a polysaccharide selected from hydroxypropyl methylcellulose (HPMC), hydroxyethyl cellulose, hydroxymethyl cellulose, D-galactopyranoside, or a derivative thereof.

6. The pH responsive material of claim 1, wherein the anchor material is a solid support formed from one or more of cotton, paper, filter paper, a chip, a pin, a slide, a membrane, a bead, a cotton swab, a wound dressing, or a particle.

7. The pH responsive material of claim 1, wherein the anchor material is a solid support comprising a cellulose particle formed from a particle size of up to about 1, 2, 3, 4, 5, 6, 7, 8, 9, 10, 15, 20, 25, 30, 35, 40, 45, 50, 60, 70, 80, 90, or 100 μm.

8. The pH responsive material of claim 1, wherein the linking moiety is a siloxane monomer or polymer thereof that comprises one or more monomers selected from GPTMS, Diethoxy(3-glycidyloxypropyl)methylsilane, 3-Glycidoxypropyldimethoxymethylsilane, 2-(3,4-epoxycyclohexyl)ethyltriethoxysilane, 2-(3,4-epoxycyclohexyl)ethyltrimethoxysilane, or combinations thereof.

9. The pH responsive material of claim 1, wherein the pH responsive element presents a visible color change at a pH of about 7.0.

10. The pH responsive material of claim 1, wherein the pH responsive element is selected from thymol blue, bromophenol blue, bromocresol green, bromocresol purple, bromothymol blue, phenol red, napththolpthalein, cresol red, cresolpthalein, phenolphthalein, or thymolpthalein.

11. The pH responsive material of claim 1, wherein the pH responsive element is a pH indicator comprising multiple phenol functional groups.

12. A pH responsive material having the structure: 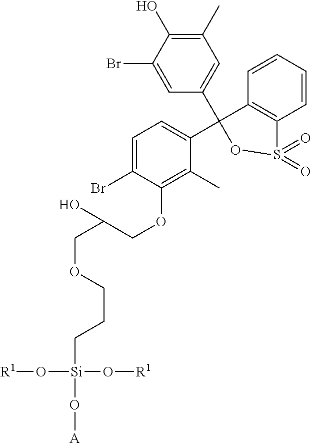
wherein;
A is cellulose or a cellulose polymer; and
each R¹ is independently H or one or more functionalized or non-functionalized GPTMS monomers or a combination thereof.

13. The pH responsive material of claim 12, wherein the cellulose or cellulose polymer is a cellulose particle having a particle size of about 20 μm.

14. The pH responsive material of claim 12, wherein the cellulose or cellulose polymer is a cellulose particle incorporated into a medical or diagnostic device by spraying, printing, or depositing the cellulose particle.

15. The pH responsive material of claim 26, wherein the cellulose or cellulose polymer is a cellulose particle sprayed, printed, or deposited on a solid phase of the medical or diagnostic device, the solid phase comprising a dressing, a wound dressing, a bandage, filter paper, or a test strip.

16. A method of making a pH responsive material comprising the steps of:
dissolving a linking moiety in an acidic solution;
immersing an anchor material in the acidic solution;
drying the anchor material; and
soaking the anchor material in a solution comprising a pH responsive element.

17. The method of claim 16, wherein the linking moiety is a silane reagent comprising an epoxide functional group.

18. The method of claim 16, further comprising a silane reagent selected from GPTMS, Diethoxy(3-glycidyloxypropyl)methylsilane, 3-Glycidoxypropyldimethoxymethylsilane, 2-(3,4-epoxycyclohexyl)ethyltriethoxysilane, 2-(3,4-epoxycyclohexyl)ethyltrimethoxysilane, or combinations thereof.

19. The method of any of claim 16, wherein the anchor material is immersed in the acidic solution for at least about 5, 10, 15, 20, 25, or 30 minutes.

20. The method of any of claim 16, wherein the acidic solution is at a temperature of at least about 60, 70, 80, 90, 100, 110, or 120° C.
